(19) United States
(12) Patent Application Publication
(10) Pub. No.: US 2021/0401197 A1
(43) Pub. Date: Dec. 30, 2021

(54) Therapeutic Pillow and Method of Manufacture Thereof

(71) Applicant: PAMELA JANE HOGSTAD, FARGO, ND (US)

(72) Inventor: PAMELA JANE HOGSTAD, FARGO, ND (US)

(21) Appl. No.: 17/342,620

(22) Filed: Jun. 9, 2021

Related U.S. Application Data
(60) Provisional application No. 63/043,538, filed on Jun. 24, 2020.

(51) Int. Cl. A47G 9/10

(52) U.S. Cl. CPC A47G 9/1081; A47G 2009/1018

(57) ABSTRACT

The present disclosure provides a specially adapted pillow, and method of manufacture thereof, which compensates for the difference in height between the lower shoulder and neck/head of a user resting on their side by providing a centrally located indent which spans the entire thickness of the pillow and which is configured to receive the lower shoulder of a user, thereby, maintaining a greater difference in height between shoulder support and neck support. The pillow may be provided as a therapeutic pillow for sufferer's of vertigo and neck pain.

### CROSS REFERENCES TO RELATED APPLICATIONS

The present application claims the benefit and priority of U.S. provisional application No. 63/043,538, filed Jun. 24, 2020.

### FIELD OF INVENTION

The present invention relates generally to the field of therapeutic pillows. More specifically, the present invention relates to a pillow having a centrally located indent for accommodating a shoulder.

### BACKGROUND

Various conditions such as back pain, back stiffness, sleep apnea, insomnia, snoring, and in particular neck pain, neck stiffness, and vertigo may be associated with, or aggravated by, an inappropriate positioning of the head and/or neck during sleep.

A pillow that is too thick or too stiff can cause strained anatomical and structural positioning of the bony, muscular, ligamentous, neurological, and vascular integrated systems of the human body, which can result in neck or back pain and obstruction to the airway.

Conversely, a pillow that is too soft and does not provide sufficient support to the head can cause the head to angle upward, which may strain the neck muscles, reduce airflow through the respiratory system, and cause headaches and neck pain. Such pillows also deform and change shape when pressure is applied, forcing users who suffer conditions and need proper head support to constantly reform the pillow into a desired shape by “fluffing” or molding it.

Orthopedic pillows have been designed to facilitate proper alignment of various parts of the body during sleep. An orthopedic pillow may have a defined shape or be formed of a particular material intended to facilitate proper alignment. However, there are few such pillows that adequately compensate for the gap between the mattress and the natural resting position of the head and neck of a user sleeping on their side. When a person lies in bed sideward, an ordinary pillow usually cannot provide sufficient support for the head and neck to rest at a right posture due to the positioning of the person's shoulder and the height different between the resting head position and the shoulder.

The cervical vertebrae will be bent in an unhealthy way. It will also produce pressure on the vertebral nerve and may result in poor quality sleep and sore of muscles in the neck and shoulder mentioned above.

While some orthopedic pillows claim to help with symptoms of vertigo and neck pain, such as contour and memory foam supports, these solutions do not allow for adjustment as the filler provided therein is not movable within the pillow. Ergonomic shape and height adjustment are essential in order to address users suffering from conditions such as neck pain and vertigo and to improve sleep quality in general also.

Furthermore, while many “orthopedic” pillow solutions already exist, they are often complex in design and expensive to manufacture, increasing the price and reducing access for some users.

It is within this context that the present invention is provided.

### SUMMARY

The present disclosure provides a specially adapted pillow, and method of manufacture thereof, which compensates for the difference in height between the lower shoulder and neck/head of a user resting on their side by providing a centrally located indent which spans the entire thickness of the pillow and which is configured to receive the lower shoulder of a user, thereby, maintaining a greater difference in height between shoulder support and neck support. The pillow may be provided as a therapeutic pillow for sufferer's of vertigo and neck pain.

Thus, according to one aspect of the present disclosure there is provided a therapeutic pillow, the pillow comprising a sewn fabric enclosure filled with a filler material, the sewn fabric enclosure being shaped to from an elongated shape having a length, a width, and a thickness, and to have an indented portion located halfway along the length and spanning the entirety of the thickness to receive the shoulder of a user having their head placed upon the pillow.

In some embodiments, the filler material comprises one or more of: foam chips, feathers, synthetic polymer, down, memory foam, and solid latex.

In some embodiments, the length, width, and thickness of the pillow are tailored to a specific user's body measurements.

In some embodiments, the density of the pillow is tailored to a specific user's therapeutic needs.

In some embodiments, the indent is in the shape of a slit.

According to another aspect of the present disclosure, there is provided a method of manufacture of a therapeutic pillow, the method comprising sewing one or more fabric pieces into a fabric enclosure shaped to have a centrally located indent for receiving a shoulder and filling the fabric enclosure with one or more filler materials.

### BRIEF DESCRIPTION OF THE DRAWINGS

Various embodiments of the invention are disclosed in the following detailed description and accompanying drawings.

FIG. 1 illustrates a first frontal isometric view of an example configuration of the disclosed pillow.

FIG. 2 illustrates a second top-down isometric view of the example configuration of the disclosed pillow with the inner filling shown within the fabric enclosure.

FIG. 3 illustrates a third side-on isometric view of the example configuration of the disclosed pillow.

FIG. 4 illustrates the third isometric view of the example configuration of the disclosed pillow with a user in position resting upon it.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

No pillow of the prior art allows a user's shoulder to be unimpeded in resting on the mattress while fully supporting the head at a natural height. Most standard pillows require the shoulder of the user to lie partially on the pillow itself because the edges are narrower leading to an insufficient height difference between shoulder and neck support when the user lies on their side. Orthopaedic solutions such as memory foam pillows are solid and cannot be adjusted.

The present disclosure provides an improved and easy to manufacture therapeutic pillow which compensates for the difference in resting height between the shoulder and head/neck of a user when lying on their side by incorporating a centrally located indent along the length of the pillow.

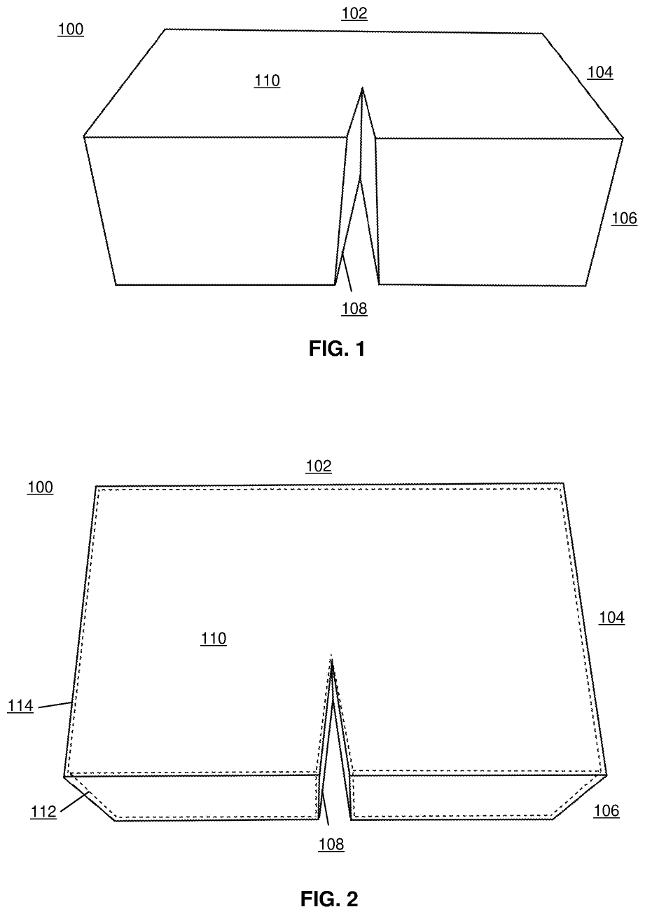

Referring to FIG. 1, a first frontal isometric view of an example configuration of the disclosed pillow 100 is shown, the pillow having a length 102, a width 104 and a thickness 106.

As mentioned, the pillow 100 further comprises an indent 108 located centrally halfway along the length 102, and spanning the entire thickness 106. The indent 108 is thus configured to receive the shoulder of a user sleeping on their side so that their lower shoulder rests on a mattress underneath while their head is supported by a top surface 110 of the pillow, thus increasing the difference in height between shoulder and neck support and alleviating neck strain and reducing the risk of vertigo dizziness.

Indent 108 is illustrated in the present example as a slit shape, which may deform to properly receive the user's shoulder, however it could take on any other suitable shape. Furthermore, while it is essential that the indent 108 is placed proximal to the centre of the pillow, it does not need to be exactly central. The term “central” as used herein means proximal to the centre.

The pillow 100 may also be made to be deeper than standard pillows. By increasing the thickness 106 of the pillow 100 and allowing the shoulder to rest on the mattress while the head is fully positioned over the pillow, the head and neck are held in a raised but natural position, reducing strain on the cervical vertebrae and alleviating the dizzying effects that many sufferers of vertigo suffer on a nightly basis due to incorrect head support.

Referring to FIG. 2, a second top-down isometric view of the example configuration of the disclosed pillow 100 is shown with the inner filling 112 shown within the fabric enclosure 114 by a dashed line.

As can be seen, there are no narrow edges that reduce the difference in height between head support and neck support and the filler 112 used is firm enough to provide sufficient support while also being adjustable.

The disclosed pillow construction includes thread (not illustrated) for sewing the fabric enclosure 114, which may be a cotton, polyester, or other type of material, and the filler material 112 which may comprise one or a combination of: foam chips, feathers, synthetic polymer, down, memory foam, and solid latex.

The pillow 100 is simple in its construction and thus easy to manufacture. Furthermore, the pillow itself resembles a standard pillow in appearance, reducing issues of a user being embarrassed of having a clearly therapeutically designed pillow on their bed.

The pillow can be adjusted to the specific needs of a user to account for their size, body type, and therapeutic needs by adjusting the overall size, height, length, width, and density of filler materials.

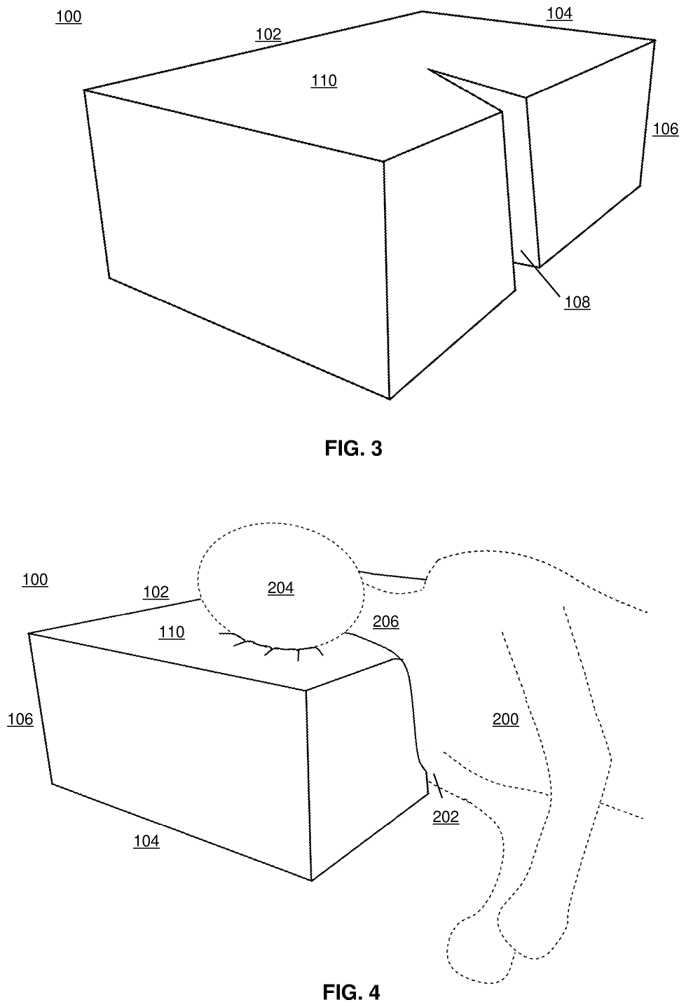

Referring to FIG. 3, a third side-on isometric view of the example configuration of the disclosed pillow 100 is shown.

Referring to FIG. 4, the third isometric view of the example configuration of the disclosed pillow 100 is shown with a user 200 in position resting upon it in a sideways resting position.

As can be seen, the lower shoulder 202 of the user 200 has been received by the indent 108 which has deformed slightly around the shoulder, and is actually resting on a surface which is supporting the pillow. This could for example be a mattress. The head 204 and neck 206 of the user are being fully supported at an elevated height by the top surface 110 of the pillow 100.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the pillow and method of manufacture have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A therapeutic pillow, the pillow comprising a sewn fabric enclosure filled with a filler material, the sewn fabric enclosure being shaped to from an elongated shape having a length, a width, and a thickness, and to have an indented portion located halfway along the length and spanning the entirety of the thickness to receive the shoulder of a user having their head placed upon the pillow.

2. A therapeutic pillow according to claim 1, wherein the filler material comprises one or more of: foam chips, feathers, synthetic polymer, down, memory foam, and solid latex.

3. A therapeutic pillow according to claim 1, wherein the length, width, and thickness of the pillow are tailored to a specific user's body measurements.

4. A therapeutic pillow according to claim 1, wherein the density of the pillow is tailored to a specific user's therapeutic needs.

5. A therapeutic pillow according to claim 1, wherein the indent is in the shape of a slit.

6. A method of manufacture of a therapeutic pillow according to claim 1, the method comprising sewing one or more fabric pieces into a fabric enclosure shaped to have a centrally located indent for receiving a shoulder and filling the fabric enclosure with one or more filler materials.
